(19) United States
(12) Patent Application Publication
(10) Pub. No.: US 2022/0057906 A1
(43) Pub. Date: Feb. 24, 2022

(54) MEDICAL DEVICE AND METHOD FOR CLEANING A TOUCHSCREEN DISPLAY

(71) Applicant: GE Precision Healthcare LLC, Wauwatosa, WI (US)

(72) Inventors: Heinz Schmied, Mondsee (AT); Andreas Haas, Schoerfling (AT)

(21) Appl. No.: 16/998,473

(22) Filed: Aug. 20, 2020

(51) Int. Cl. G06F 3/0488; A61B 8/00

(52) U.S. Cl. CPC G06F 3/0488; A61B 8/4483; A61B 8/461

(57) ABSTRACT

Various methods and system are provided for cleaning a touchscreen display that is part of a medical device. In one example, a method includes entering a cleaning mode in response to a user input, receiving cleaning touch inputs through the touchscreen display while in the cleaning mode, and graphically representing an area of the touchscreen display that has been contacted with the cleaning touch inputs in real-time while in the cleaning mode to illustrate the area of the touchscreen display that has been cleaned.

### FIELD

Embodiments of the subject matter disclosed herein relate to a medical device and a method for cleaning a touchscreen display that is part of the medical device.

### BACKGROUND

Conventional medical devices often include a touchscreen display that is used both for displaying information and as an input device for receiving touch-based commands. The touchscreen display may be used to display a graphical user interface, patient information, patient vitals, and/or medical images. It may be desirable to have a clean touchscreen display both for reasons of improved usability and for safety and hygiene. With conventional medical devices that include a touchscreen display, it can be difficult for the user to accurately determine whether or not all of the touchscreen display has been adequately cleaned. For at least these reasons, there is a need for an improved method and medical device to enable easier cleaning of a touchscreen display.

### BRIEF DESCRIPTION

In one embodiment, a method for cleaning a touchscreen display that is part of a medical device includes entering a cleaning mode in response to a user input, receiving cleaning touch inputs through the touchscreen display while in the cleaning mode, and graphically representing an area of the touchscreen display that has been contacted with the cleaning touch inputs in real-time while in the cleaning mode to illustrate the area of the touchscreen display that has been cleaned.

In one embodiment, a medical device includes a touchscreen display, a memory, and a processor. The processor is configured to control the touchscreen display to enter a cleaning mode in response to a user input. The processor is configured to graphically represent an area of the touchscreen display that has been contacted with cleaning touch inputs in real-time while in the cleaning mode to illustrate the area of the touchscreen display that has been cleaned.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example of a medical device according to an embodiment;

FIG. 2 shows and example of an ultrasound imaging system according to an embodiment;

FIG. 3 shows a flow chart illustrating an examplary method according to an embodiment;

FIG. 4 shows and example of a cleaning mode image that may be displayed on a touchscreen display in a cleaning mode in accordance with an embodiment;

FIG. 5 shows and example of a cleaning mode image that may be displayed on a touchscreen display in a cleaning mode in accordance with an embodiment;

FIG. 6 shows an example of a screenshot that may be displayed on a touchscreen display while cleaning the touchscreen display in accordance with an embodiment; and

FIG. 7 shows an example of screenshot that may be displayed on a touchscreen display while cleaning the touchscreen display in accordance with an embodiment.

### DETAILED DESCRIPTION

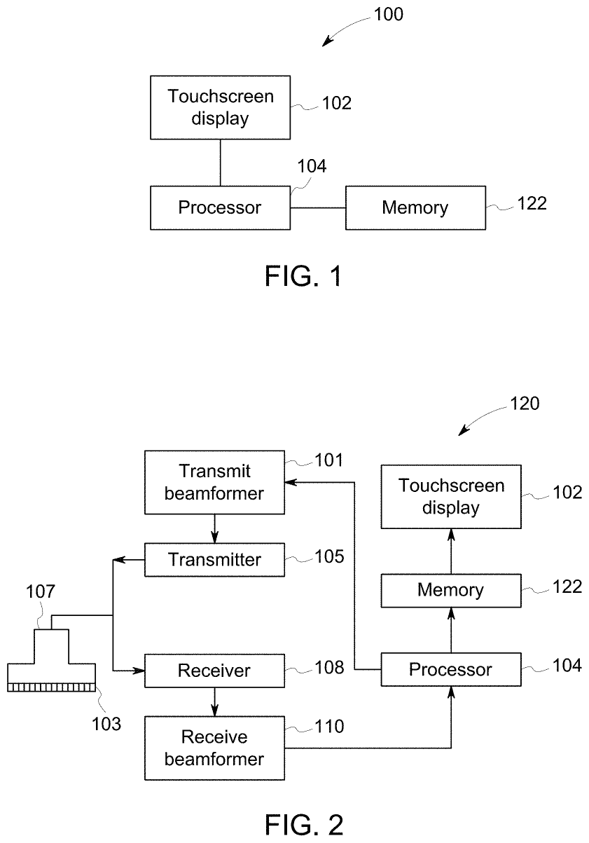

FIG. 1 is schematic representation of a medical device 100 in accordance with an embodiment. The medical device 100 includes a touchscreen display 102, a processor 104, and a memory 122. The touchscreen display 102 is configured to operate as both an input and an output device. The touchscreen display 102 may, for instance, include both a display portion or layer and a touch-sensitive portion or layer. The display portion may, for example, be a Light-Emitting Diode (LED) display or an Organic Light-Emitting Diode (OLED) display. The touch-sensitive portion of the touchscreen display 102 may use capacitive sensing, resistive sensing or other technologies in order to detect a user's single-touch or multi-touch inputs on the touchscreen display 102. The touchscreen display 102 enables a user to input gestures or interact with a graphical user interface (GUI) displayed on the display portion of the touchscreen display 102. The touchscreen display 102 may be configured to display data such as images, GUIs, menus, patient information, etc. Touchscreen displays are well known by those skilled in the art and will therefore not be described in additional detail.

The processor 104 controls the data that is displayed on the touchscreen display 102 and receives commands that are inputted through the touchscreen display 102. The processor 104 may include one or more other electronic components capable of carrying out processing functions, such as one or more digital signal processors, field-programmable gate arrays, graphic boards, and/or integrated circuits. According to other embodiments, the processor 104 may include multiple electronic components capable of carrying out processing functions. Other embodiments may use two or more separate processors to perform the functions performed by the processor 104 described with respect to FIG. 1. The memory 122 may comprise any known data storage medium, such as one or more tangible and non-transitory computer-readable storage media (e.g., one or more computer hard drives, disk drives, universal serial bus drives, solid-state drives, or the like). The memory 122 is configured to store executable instructions that may be executed by the processor 104.

FIG. 2 is a schematic of an ultrasound imaging system 120 according to an exemplary embodiment. The ultrasound imaging system 120 includes the touchscreen display 102, the processor 104, and the memory 122 that were previously described with respect to FIG. 1

The ultrasound imaging system 120 includes a transmit beamformer 101 and a transmitter 105 that drive elements 103 within an ultrasound probe 107 to emit pulsed ultrasonic signals into a body (not shown). According to an embodiment, the ultrasound probe 107 may be a linear probe, a curvilinear probe, a phased array probe, a linear phased array probe, a curvilinear phased array probe, a two-dimensional matrix array probe, a curved two-dimensional matrix array probe, a mechanical 3D probe, or any other type of ultrasound probe capable of acquiring diagnostic ultrasound images.

The pulsed ultrasonic signals are back-scattered from structures in the body, such as blood cells or muscular tissue, to produce echoes that return to the elements 103. The echoes are converted into electrical signals by the elements 103, and the electrical signals are received by a receiver 108. The electrical signals representing the received echoes are passed through a receive beamformer 110 that outputs ultrasound image data. The ultrasound probe 107 may contain electronic circuitry to do all or part of the transmit and/or the receive beamforming. For example, all or part of the transmit beamformer 101, the transmitter 105, the receiver 108, and the receive beamformer 110 may be situated within the ultrasound probe 107 in other embodiments. Scanning may include acquiring data through the process of transmitting and receiving ultrasonic signals. Ultrasound image data acquired by the ultrasound probe 107 can include one or more datasets acquired with the ultrasound imaging system 100.

The processor 104 may be further configured to control the transmit beamformer 101, the transmitter 105, the receiver 108, and the receive beamformer 110. The processor 104 is in electronic communication with the ultrasound probe 107 via one or more wired and/or wireless connections. The processor 104 may control the ultrasound probe 107 to acquire data. The processor 104 controls which of the elements 103 are active and the shape of a beam emitted from the ultrasound probe 107. The processor 104 is also in electronic communication with the touchscreen display 102. The processor 104 may be configured to display images generated from the ultrasound image data on the touchscreen display 102 or the processor 104 may be configured to display images generated from the ultrasound image data on a separate display device (not shown on FIG. 2). For example, according to an exemplary embodiment, the touchscreen display 102 may be used primarily as a user interface device and the images generated from the ultrasound image data may be displayed primarily on one or more separate display devices. The processor 104 may include one or more central processors according to an embodiment. According to other embodiments, the processor 104 may include one or more other electronic components capable of carrying out processing functions, such as one or more digital signal processors, field-programmable gate arrays, graphic boards, and/or integrated circuits. According to other embodiments, the processor 104 may include multiple electronic components capable of carrying out processing functions. Other embodiments may use two or more separate processors to perform the functions performed by the processor 104 according to the exemplary embodiment shown in FIG. 1. According to another embodiment, the processor 104 may also include a complex demodulator (not shown) that demodulates the radio frequency data and generates raw data. In another embodiment, the demodulation can be carried out earlier in the processing chain.

The processor 104 is adapted to perform one or more processing operations according to a plurality of selectable ultrasound modalities on the data. The data may be processed in real-time during a scanning session as the echo signals are received, such as by processing the data without any intentional delay, or processing the data while additional data is being acquired during the same imaging session of the same person.

The data may be stored temporarily in a buffer (not shown) during a scanning session and processed in less than real-time in a live or off-line operation. Some embodiments of the inventive subject matter may include multiple processors (not shown) to handle the processing tasks that are handled by the processor 104 according to the exemplary embodiment described hereinabove. For example, a first processor may be utilized to demodulate and decimate the RF signal while a second processor may be used to further process the data prior to displaying an image. It should be appreciated that other embodiments may use a different arrangement of processors.

The ultrasound imaging system 120 may continuously acquire ultrasound data at a frame-rate of, for example, 10 to 30 hertz. Images generated from the data may be refreshed at a similar frame-rate. Other embodiments may acquire and display ultrasound data at different rates. For example, some embodiments may acquire ultrasound data at a frame-rate of less than 10 hertz or greater than 30 hertz.

The memory 122 is included for storing processed frames of acquired data. In one embodiment, the memory 122 is of sufficient capacity to store at least several seconds worth of ultrasound image data. The frames of data are stored in a manner to facilitate retrieval thereof according to their order or time of acquisition. The memory 122 may also be used to store executable instructions that may be executed by the processor 104.

In various embodiments of the present invention, data may be processed by other or different mode-related modules by the processor 104 (e.g., B-mode, Color Doppler, M-mode, Color M-mode, spectral Doppler, Elastography, TVI, strain, strain rate, and the like) to form two- or three-dimensional image data. For example, one or more modules may generate B-mode, color Doppler, M-mode, color M-mode, spectral Doppler, Elastography, TVI, strain, strain rate and combinations thereof, and the like. Timing information indicating a time at which the data was acquired in memory may be recorded. The modules may include, for example, a scan conversion module to perform scan conversion operations to convert the image volumes from beam space coordinates to display space coordinates. A video processor module may read the image frames from a memory and displays an image in real time while a procedure is being carried out on a person. A video processor module may store the images in an image memory, from which the images are read and displayed.

While FIG. 2 shows an exemplary embodiment where the medical device is an ultrasound imaging system, the medical device may be a different type of medical imaging system according to various embodiments. For example, the medical device may be an X-ray imaging system, a Computed Tomography (CT) imaging system, a Positron Emission Tomography (PET) imaging system, a Single Photon Emission Computed Tomography (SPECT) imaging system, or a Magnetic Resonance (MR) imaging system. According to other embodiments, the medical device may be non-imaging medical device. For example, the medical device may be a patient monitoring device for monitoring parameters of the patient such as blood pressure, heart rate, respiratory rate, etc. According to other embodiments, the medical device may be a patient support device such as a ventilator or an infant warmer.

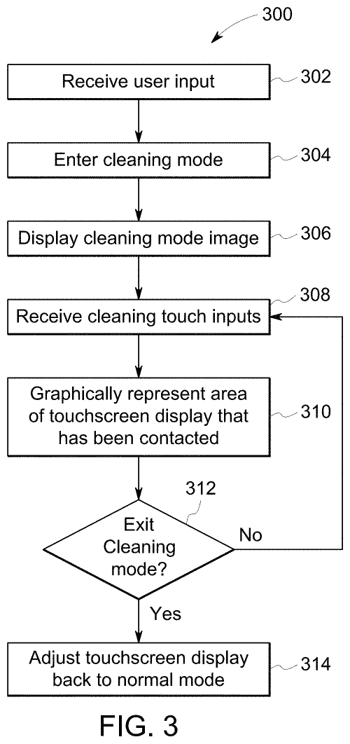

A flow chart is shown in FIG. 3, illustrating a method 300 for cleaning a touchscreen display of a medical device. The method 300 may be implemented as executable instructions in non-transitory memory, such as memory 122, and executed by a processor, such as processor 104. The technical effect of the method 300 is graphically representing an area of the touchscreen display that has been cleaned while in a cleaning mode.

Referring to the method 300 in FIG. 3, at step 302, the processor 104 receives a user input to enter a cleaning mode. According to an embodiment, the user input may be entered through the touchscreen display 102, such as by selecting a button to enter the cleaning mode. For example, the user may click on a control or button labeled “Cleaning Mode”. The user may optionally select the control to enter the cleaning mode through other control inputs such as by accessing a drop-down menu. According to other embodiments, the user input to enter the cleaning mode may be entered through a different control input. For example, the user may enter the command to enter the cleaning mode through a different user interface device that is located on the medical device in a location that is separate from the touchscreen display 102.

At step 304, the processor causes the medical device 100 to enter a cleaning mode in response to the user input received at step 302. At optional step 306, the processor 104 may automatically display a cleaning mode image on the touchscreen display 102 in response to entering the cleaning mode. According to an embodiment the cleaning mode image may be an image that is either a uniform first color or that is substantially a uniform first color. For example, according to an embodiment where the cleaning mode image is substantially a uniform first color, all of the cleaning mode image may be the first color, such as black, except for a portion of the cleaning mode image that includes instructions for exiting the cleaning mode. According to an embodiment where the cleaning mode image is a uniform first color, all of the cleaning mode image may be the first color, such as black. According to other embodiments, the cleaning mode image may be all a uniform first greyscale value or substantially all a uniform first greyscale value. For example, according to an embodiment where the cleaning mode image is substantially all the first uniform greyscale value, all of the cleaning mode image may be the first greyscale value except for a portion of the cleaning mode image that includes instructions for exiting the cleaning mode. According to other embodiments, the cleaning mode image may be all the uniform first greyscale value. The cleaning mode image is configured to fill all of the display area of the touchscreen display 102.

Configuring the processor 104 to automatically adjust the touchscreen display 102 to display a cleaning mode image that is a dark color, such as black, provides the advantage of making it easier for the user to see dust, dirt, or smudges on the touchscreen display 102. It is generally easier to identify dust, dirt, or smudges against a dark background such as a black screen. Adjusting the touchscreen display 102 to display a uniform or substantially uniform background on the touchscreen display in the cleaning mode makes it easier for the user to see areas of dirt or dust on the touchscreen display 102.

At step 308, cleaning inputs are received through touchscreen display 102. According to an embodiment, cleaning inputs may include wiping the touchscreen display 102 with a cloth or other cleaning device such as sponge. According to some embodiments, the cloth or other cleaning device may be used to apply a cleaning solution and/or disinfectant to the touchscreen display 102.

Next, at step 310, the processor 104 controls the touchscreen display 102 to graphically represent an area of the touchscreen display 102 that has been contacted while in the cleaning mode. At step 312, the processor 104 determines if a user has provided a command to exit the cleaning mode. If a command to exit the cleaning mode has not been received, the method 300 returns to step 308 where additional cleaning touch inputs are received. The method 300 iteratively repeats steps 308, 310, and 312 until either a command to exit the cleaning mode has been received or the user stops providing cleaning touch inputs at step 308. It is anticipated that once the user is done providing cleaning touch inputs, the user will enter a command to exit the cleaning mode.

As the method 300 iteratively repeats steps 308, 310, and 312, the processor 104 graphically represents the area of the touchscreen display 102 that has been contacted in real-time while in the cleaning mode. In other words, the processor 104 updates the touchscreen display 102 as the user provides cleaning touch inputs so that the graphical representation of the area of the touchscreen display that has been contacted is accurate and up-to-date in real-time as the user is providing the cleaning touch inputs.

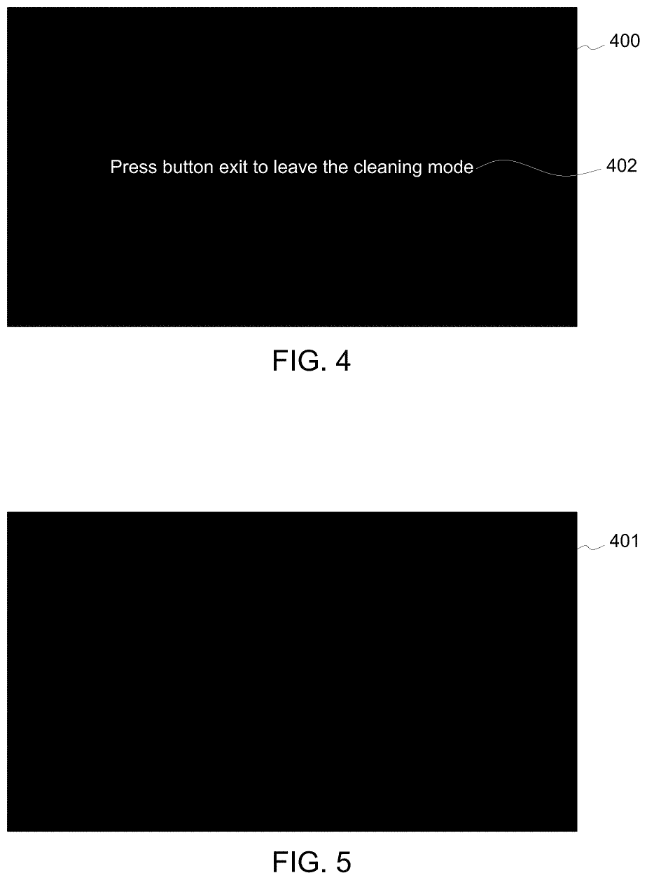

FIG. 4 is a representation of a cleaning mode image 400 according to an embodiment. In FIG. 4, the cleaning mode image 400 is substantially black, but includes a text string 402 providing an instruction for exiting the cleaning mode. Embodiments may display instructions for leaving the cleaning mode differently. Instructions for leaving the cleaning mode may be displayed after a fixed amount of time in the cleaning mode or only once the processor 104 has determined that all of the surface of the touchscreen display has been cleaned. In other embodiments, the cleaning mode image may not include instructions for exiting the cleaning mode. FIG. 5 is a representation of a cleaning mode image 401 according to an embodiment. In FIG. 5, the cleaning mode image 401 may be displayed on the touchscreen display 120 in response to entering the cleaning mode in according with an embodiment. In FIG. 5, the first color is black. In the cleaning mode image 401, all of the touchscreen display 102 is black, but other colors may be used for the cleaning mode image 401 according to various embodiments.

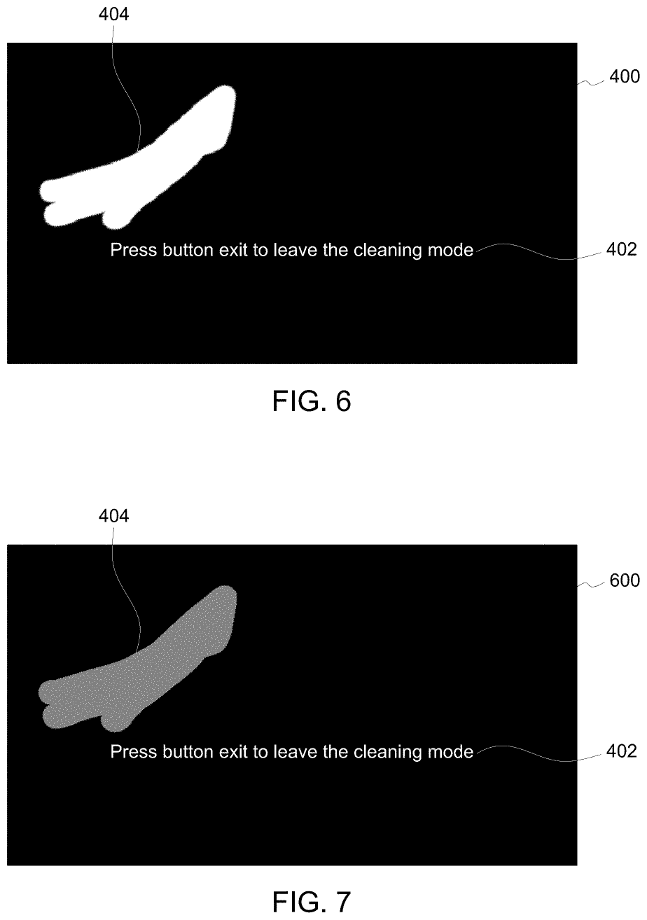

FIG. 6 is a screenshot of the touchscreen display 102 in accordance with an embodiment. FIG. 6 includes a graphical representation of a clean region 404. The clean region 404 displayed on the touchscreen display 102 represents the area or region of the touchscreen display 102 that has been contacted while in the cleaning mode. In FIG. 6, the clean region 404 is shown in a second color, white, that is different than the first color, which is black in the example shown in FIG. 6. According to other embodiments, the processor 104 may use colors other than white to provide a graphical representation of the clean region 404 on the touchscreen display 102. For embodiments using color to graphically represent the clean region 404, the second color should have a different hue than the first color in order to clearly show the extent of the clean region 404. It may be desirable to use a second color that is a lighter hue than the first color in order to make the clean region 404 easy to identify. For embodiments using different greyscale values to graphically represent the clean region 404, a second greyscale value, different than the first greyscale value may be used to clearly show the extent of the clean region 404. It may be desirable to use a second greyscale value that is lighter than the first greyscale value to clearly show the extent of the clean region 404. For some medical devices, ensuring that the touchscreen display is adequately cleaned and disinfected may be a matter of patient and/or operator safety.

According to other embodiments, at step 306, the first color used for the cleaning mode image may be a color other than black. For example, the processor 104 may control the touchscreen display 102 to display a cleaning mode image that is a different color or a different greyscale value.

FIG. 7 is a screenshot 600 of the touchscreen display according to an embodiment where a different greyscale value is used to indicate the clean region 404. The first greyscale value is darker than the second greyscale value used to indicate the clean region 404.

By iteratively performing steps 308, 310, and 312 of the method 300 while the user applies cleaning inputs to the touchscreen display 102, the processor 104 is able to graphically represent the area of the touchscreen display 102 that has been contacted, and therefore cleaned, while in the cleaning mode. According to an exemplary embodiment, the processor 104 may be configured to update the graphical representation of the clean region 404 as the user is cleaning the touchscreen display. This allows for the size and configuration of the clean region 404 to be updated in order to represent the size and configuration of the clean region in real-time as the user cleans the touchscreen display 102. According to any example, the processor 104 may be configured to iteratively perform steps 308, 310, and 312 multiple times each second, such as at a rate of greater than 5 Hz. This provides the user with a very accurate real-time indication of the clean region 404 and the region that still needs to be cleaned. Providing a real-time graphical representation of the clean region 404 of the touchscreen display 102 that has been cleaned and the area of the touchscreen display 102 left to be cleaned helps to ensure a more thorough cleaning of the touchscreen display 102. For clinical situations where cleanliness is important to patient and or clinician safety, providing a graphical representation of the clean region helps to ensure a cleaner and therefore safer medical device.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for cleaning a touchscreen display that is part of a medical device, the method comprising:
entering a cleaning mode in response to a user input;
receiving cleaning touch inputs through the touchscreen display while in the cleaning mode; and
graphically representing an area of the touchscreen display that has been contacted with the cleaning touch inputs in real-time while in the cleaning mode to illustrate the area of the touchscreen display that has been cleaned.

2. The method of claim 1, wherein said graphically representing the area of the touchscreen display that has been contacted while in the cleaning mode comprises colorizing the area that has been contacted while in the cleaning mode on the touchscreen display.

3. The method of claim 2, further comprising automatically displaying a cleaning mode image on the touchscreen display in response to receiving the user input to enter the cleaning mode, wherein the cleaning mode image is substantially a uniform first color, and wherein said colorizing the area comprises displaying the area that has been contacted while in the cleaning mode with a second color that is different than the first color.

4. The method of claim 3, wherein the second color is a lighter hue than the first color.

5. The method of claim 4, wherein the first color is black.

6. The method of claim 1, wherein said graphically representing the area of the touchscreen display that has been contacted while in the cleaning mode comprises adjusting a greyscale value of the area of the touchscreen display that has been contacted while in the cleaning mode.

7. The method of claim 2, further comprising automatically displaying a cleaning mode image on the touchscreen display in response to receiving the user input to enter the cleaning mode, wherein the cleaning mode image is substantially a uniform first greyscale value, and wherein said graphically representing the area comprises displaying the area that has been contacted while in the cleaning mode with a second greyscale value that is different than the first greyscale value.

8. The method of claim 7, wherein the first greyscale value is darker than the second greyscale value.

9. The method of claim 8, wherein the first greyscale value is black and the second greyscale value is white.

10. The method of claim 3, wherein the cleaning mode image includes an instruction for exiting the cleaning mode on the touchscreen display

11. The method of claim 1, further comprising detecting that all of the touchscreen display has been contacted with cleaning touch inputs while in the cleaning mode and displaying an indicator that cleaning has been completed.

12. The method of claim 1, wherein the medical device is an ultrasound imaging system.

13. A medical device comprising:
a touchscreen display;
a memory; and
a processor;
wherein the processor is configured to control the touchscreen display to enter a cleaning mode in response to a user input; and
wherein the processor is configured to graphically represent an area of the touchscreen display that has been contacted with cleaning touch inputs in real-time while in the cleaning mode to illustrate the area of the touchscreen display that has been cleaned.

14. The medical device of claim 13, wherein the processor is configured to graphically represent the area of the touchscreen display that has been contacted while in the cleaning mode by colorizing the area of the touchscreen display.

15. The medical device of claim 14, wherein the processor is configured to display a cleaning mode image on the touchscreen display in response to receiving the user input to enter the cleaning mode, wherein the cleaning mode image is substantially a uniform first color, and wherein said colorizing the area comprises displaying the area that has been contacted while in the cleaning mode with a second color that is different than the first color.

16. The medical device of claim 15, wherein the second color is a lighter hue than the first color.

17. The medical device of claim 13, wherein the processor is configured to graphically represent the area of the touchscreen display that has been contacted while in the cleaning mode by adjusting a greyscale value of the area of the touchscreen.

18. The medical device of claim 17, wherein the processor is configured to display a cleaning mode image on the touchscreen display in response to receiving the user input to enter the cleaning mode, wherein the cleaning mode image is substantially a first greyscale value, and wherein said adjusting the greyscale value comprises displaying the area that has been contacted while in the cleaning mode with a second greyscale value that is different than the first greyscale value.

19. The medical device of claim 18, wherein the first value is darker than the second value.

20. The medical device of claim 13, further comprising:
an ultrasound probe;
a transmitter;
a transmit beamformer; a receiver; and
a receive beamformer.
